(19) United States
(12) Patent Application Publication
(10) Pub. No.: US 2002/0028520 A1
(43) Pub. Date: Mar. 7, 2002

(54) Multifunctional ceramic particles as solid supports for solid phase and combinatorial solid phase synthesis

(76) Inventors: Egisto Boschetti, Croissy sur Seine (FR); Petr Kocis, Boothwyn, PA (US)

Correspondence Address: Stephen A. Bent, FOLEY & LARDNER, Washington Harbour, 3000 K Street, N.W., Suite 500, Washington, DC, 20007-5109, US

(21) Appl. No.: 09/741,146

(22) Filed: Dec. 21, 2000

Related U.S. Application Data
(63) Continuation of application No. 08/820,563, filed on Mar. 19, 1997, now abandoned.

(51) Int. Cl. G01N 33/543; B01J 20/26

(52) U.S. Cl. 436/518; 502/402

(57) ABSTRACT

Inert, thermally and mechanically stable porous ceramic solid phase supports for use in solid phase synthesis of molecules wherein the porous ceramic solid supports can be used under a wide variety of reaction conditions and in synthesis methods, including but not limited to organic, regular, multiple parallel, or combinatorial organic synthesis on a solid phase. Methods of using the porous ceramic solid supports for solid phase synthesis of molecules, for solid phase synthesis of polypeptides or peptidomimetics, for generating combinatorial libraries of linkers, and for combinatorial synthesis of compounds, including small molecules and aromatic and heteroaromatic compounds.

### FIELD OF THE INVENTION

The present invention relates to supports for solid phase and combinatorial solid phase synthesis of molecules, and methods of using the same.

### BACKGROUND OF THE INVENTION

The techniques used for solid phase synthesis of combinatorial libraries are based in essence on peptide solid phase synthesis which was pioneered by Merrifield, who developed partially crosslinked polystyrene beads (styrene crosslinked with 1-2% divinylbenzene) that were derivatized to allow introduction of different linkers and handles.

The invention by Merrifield of a practical and economical solid phase method of synthesizing polypeptides opened the door to a host of new synthesis methods based on solid supports. The ability to rapidly synthesize and purify complex macromolecules, such as polypeptides and polynucleotides, has made possible advances in enzymology and nucleic acids research by providing access to almost any short polypeptide or polynucleotide.

More recently, the burgeoning field of combinatorial chemistry has spurred the growth of solid phase synthesis schemes for use with large arrays of small organic molecules. For example, the ability to synthesize a large number of potential ligands or drugs, combined with methods for high throughput screening the products for efficacy, has allowed the rapid identification of active compounds from a partially or fully random mixture of candidate molecules. Examples of combinatorial synthesis and screening of candidate molecules are described, e.g., by the following references: Bunin et al., Proc. Nat'l Acad. Sci. USA, 91:4708-4712 (1994); Gordon et al, J. Med. Chem., 37:1385-1401 (1994); Kocis et al, Tetrahedron Lett., 36:6623-6626 (1995); Kurth et al., J. Org. Chem., 59:5862-5864 (1994); Lam et al., Nature, 354:82-84 (1991); Owens et al., Biochem, Biophys. Res. Commun, 181:402-408 (1991); Patek et al., Tetrahedron Lett., 35:9169-9172 (1994); Salmon et al., Proc, Nat'l Acad. Sci. USA, 90:11708-11712 (1993); Sebestyen et al., Bioorg. Med. Chem., 3:413-418 (1993); and Zuckermann et al., Proc Nat'l Acad. Sci. USA, 89:4505-4509 (1992).

One of the challenges in combinatorial chemistry is the selection of the proper solid support. When the variety of chemical reactions to which solid phase synthesis potentially may apply is compared with commercially available resins, it is readily apparent that the choice of solid supports is limited. This limited choice of solid supports also limits the chemistries which may actually be used for solid phase synthesis of combinatorial libraries, thereby limiting the chemical diversity of these combinatorial libraries Hence, there is significant need for a wider variety of solid supports for combinatorial synthesis.

Existing solid phase synthesis procedures employ a variety of solid or insoluble supports for immobilization of precursor molecules. Examples of existing solid supports include resin beads (such as polystyrene-based polymers, including chloromethyl polystyrene, aminomethylpolystyrene, 2-chlorotrityl chloride-resin, and the like), silica gel, controlled pore glass beads, and solid surfaces. In addition, improved versions of Merrifield's classical polystyrene resin are also available, such as polystyrene-polyethylene glycol graft polymers, e.g., TentaGel resins or NovaSyn TG resin (manufactured by Rapp Polymere Gmbh, Tubingen, Germany), dimethylacrylamide supported on high cross-linked polystyrene (commercially available as NovaSyn P500 from NovaBiochem, San Diego, Calif.), and dimethylacrylamide supported on Kieselguhr (commercially available as NovaSyn K125 from NovaBiochem, San Diego, Calif.).

However, most solid phase supports are not suitable for synthesis under a variety of conditions. For example, polymeric resins, such as polystyrene beads, are often chemically reactive under certain vigorous conditions, such as conditions for Friedel-Crafts acylation and alkylation, making such resins generally unsuitable for synthesis of immobilized molecules under these harsh conditions. Controlled pore glass beads, while nonreactive, can have relatively low loading capacities, and thus are often less suitable for synthesis of relatively large quantities of compounds.

An additional drawback of certain commonly-used solid supports is the relatively low mechanical stability of the support structure. For example, some kinds of resin beads are often mechanically weak, and may be deformed or crushed by high pressure, such as the pressure found in a packed column continuous-flow reactor or by mechanical handling of a resin, e.g., Kieselguhr resin. The disintegration or deformation of the beads can obstruct the flow in such a reactor, causing excessive back pressure conditions. Also, certain polymeric supports must be swollen in a solvent for best efficiency in reactions of the immobilized molecules. These polymeric supports are therefore suitable for use with only a limited number of solvents, with attendant limitations on the reactants and chemistries that can be employed. Certain polymeric supports shrink when cooled to low temperature, which may prevent homogenous reaction of the immobilized molecules, result in reduced yields, and incomplete reaction which would cause more than one structure to be attached on a bead.

The use of Kieselguhr (diatomaceous earth) particles as an inert solid substrate for polymeric resin supports has been reported (Atherton et al., J. Chem. Soc., Chem.Commun. 1151 (1981); and U.S. Pat. No. 4,735,985 to Rosevear et al.). These particles have sufficient mechanical strength to be packed into columns and used in continuous flow peptide synthesis, but are not strong enough to be used under conditions of combinatorial chemistry, especially using a split and mix method.

### SUMMARY OF THE INVENTION

In one aspect, the present invention relates to a porous ceramic solid support for use in solid phase and combinatorial solid phase synthesis of molecules comprising ceramic particles, wherein the ceramic particles comprise a ceramic surface material having exterior and interior surfaces, and pores which permeate the interior of the ceramic particles, and wherein the ceramic surface material is derivatized with one or more chemical functionalities.

In another embodiment, the present invention relates to a porous ceramic solid support for use in solid phase and combinatorial solid phase synthesis of molecules comprising pore-filled ceramic particles, wherein the pore-filled ceramic particles comprise a ceramic surface material having exterior and interior surfaces, and pores which permeate the interior of the pore-filled ceramic particles and are substantially filled with a three-dimensional, pore-filling polymer network, and wherein said three-dimensional, pore-filling polymer network is derivatized with one or more chemical functionalities The three-dimensional, pore-filling polymer network, or gel, is derived from the polymerization of a mixture comprising effective amounts of a main monomer, a passivating or neutralizing monomer different from the main monomer, and a crosslinking agent, and may optionally include either or both a pore inducing agent and a polymerization initiator.

The ceramic surface material of the porous ceramic solid supports of the present invention may be selected from the group consisting of alumina, alumina silicate, aluminium oxide, aluminium nitride, berrylia, barium titanate, fused silica, silicon carbide, silicon nitride, boron nitride, boron carbide, silicon or boron carbonitride, titanium, titanium oxide, titanium boride, titanium carbide, hafnium, hafnium oxide, cerium, cerium oxide, zirconium, zirconium oxide, yttrium, yttrium oxide, zirconia-toughened alumina, and mixtures thereof.

In certain embodiments of the present invention, the ceramic surface material of the porous ceramic solid support may be coated with a polymer to provide desirable surface properties. The polymer may, for example, generally consist of a linear, high molecular weight polymer capable of being dissolved in an organic solvent to form a coating solution. Typical concentrations of polymer in the coating solution range from about 2% (w/v) to about 20% (w/v).

In another embodiment of the present invention, the ceramic surface material of ceramic particles, or the three-dimensional pore-filling polymer network of pore-filled ceramic particles, is derivatized to contain one or more appropriate chemical functionalities comprising surface reactive groups having compatible linkers attached thereto, wherein said linker includes a chemically reactive group.

According to the present invention, chemical functionalities or chemically reactive groups which may derivatize the porous ceramic solid supports may be selected from the group consisting of amine, protected amine, carbonyl, carboxyl, activated carboxyl, hydroxyl, epoxide, anhydride, thiol, carboxamido, double bond, triple bond, sulphonyl, carboxamidohydroxyl, and aromatic and heteroaromatic moieties.

In a preferred embodiment, the ceramic particles or pore-filled ceramic particles of the porous ceramic solid support have initial average size of about 5 μm to about 3 mm, and more preferably about 10 μm to about 1 mm. The ceramic particles or pore-filled ceramic particles of the porous ceramic solid support preferably have an initial pore size ranging from about 40 Angstroms to about 6000 Angstroms, more preferably from about 500 Angstroms to about 3000 Angstroms, and even more preferably from about 800 to about 1500 Angstroms.

In another aspect, the present invention relates to a method for solid phase synthesis of molecules using a porous ceramic solid support comprising the steps of derivatizing ceramic surface material of said porous ceramic solid support with one or more appropriate chemical functionalities which permit attachment of an organic molecule to said ceramic surface material, attaching said organic molecule to the ceramic surface material, and subjecting the organic molecule to reactions which result in synthesis of said molecules.

The present invention further relates to a method for solid phase synthesis of molecules using a porous ceramic solid support, wherein said porous ceramic solid support comprises pore-filled ceramic particles, comprising the steps of derivatizing a three-dimensional polymer network within interior channels of said pore-filled ceramic particles with one or more appropriate chemical functionalities which permit attachment of an organic molecule to said three-dimensional polymer network, attaching said organic molecule to the three-dimensional polymer network, and subjecting the organic molecule to reactions which result in synthesis of said molecules.

In a further aspect, the invention relates to a method for solid phase synthesis of polypeptides or peptidomimetics using a porous ceramic solid support which comprise the steps of derivatizing ceramic surface material or three-dimensional polymer network of said porous ceramic solid support with an amino functionality, coupling a compatible or appropriate linker to said amino functionality and coupling a first amino acid or peptidomimetic moiety to said linker, coupling one or more additional amino acids or peptidomimetic moieties to said first amino acid or peptidomimetic moiety, cleaving the resulting elongated polypeptide chain or peptidomimetic product from the porous ceramic solid support, and purifying the resulting polypeptide or peptidomimetic product.

Another embodiment of the present invention relates to a method for solid phase synthesis of polypeptides or peptidomimetics using a porous ceramic solid support comprising the steps of derivatizing ceramic surface material or three-dimensional polymer network of said porous ceramic solid support with a phenyl functionality, derivatizing the phenyl functionality to yield an appropriate derivatized functionality, coupling a compatible linker to said appropriate derivatized functionality and coupling a first amino acid or peptidomimetic moiety to said linker, coupling one or more additional amino acids or peptidomimetic moieties to said first amino acid or peptidomimetic moiety, cleaving the resulting elongated polypeptide chain or peptidomimetic product from the porous ceramic solid support, and purifying the resulting polypeptide or peptidomimetic product.

Still another aspect of the invention relates to a combinatorial synthesis method for preparing aromatic and heteroaromatic compounds using a porous ceramic solid support comprising the steps of functionalizing ceramic surface material or three-dimensional polymer network of said porous ceramic solid support with one or more aromatic functionalities and compatible linkers, subjecting the aromatic functionalities to reactions which result in synthesis of said aromatic and heteroaromatic compounds, and cleaving and isolating the resulting compounds.

The skilled artisan would readily recognize the wide variety of reactions which may be used in solid phase and combinatorial solid phase synthesis methods utilizing the porous ceramic solid supports of the present invention For example, reactions which may be used in the synthesis methods of the present invention may comprise one or more reactions selected from the group consisting of Friedel-Crafts alkylation with alkyl halides, alcohols, or alkenes in the presence of Lewis acids; Friedel-Crafts acylation with acid chlorides, or acid anhydrides; formulation of the arylaldehyde via the Vilsmeier reaction using activated aromatic rings, dimethylformamide and phosphorus oxychloride; Michael addition; and transition metal mediated reactions.

A further aspect of the invention is a method for generating combinatorial libraries of molecules, including small molecules, aromatic and heteroaromatic molecules, peptides, peptidomimetics, and linkers, using a porous ceramic solid support comprising the steps of functionalizing ceramic particles or pore-filled ceramic particles of the porous ceramic solid support with chemical functionalities, which optionally may include a compatible linker, that permit the attachment of an organic molecule to said ceramic particles or pore-filled ceramic particles, attaching the organic molecule to said ceramic particles or pore-filled ceramic particles, subjecting the resulting attached organic molecule to reactions which result in synthesis of desired molecules, cleaving the desired molecules from the ceramic particles, and isolating the resulting molecules.

### DETAILED DESCRIPTION OF THE INVENTION

Combinatorial organic synthesis on a solid phase is a rapidly growing area of very intensive research effort It has become an increasingly more important method for drug lead discovery, as well as a tool for optimization and rapid structure-activity relationship studies

One of the primary limitations of existing solid supports used for solid phase organic combinatorial chemistry is that these supports contain aromatic rings in the matrix which may be affected by electrophilic aromatic substitution reactions. Thus, reactions such as halogenation, nitration or sulfonation cannot be performed on these existing resins without affecting aromatic ring of the matrix which would lead to unpredictable changes in the properties of the solid support. This is a significant drawback, as a vast number of existing drugs contain aromatic moieties.

The ceramic particles of the present invention have a non-aromatic matrix (covalently bonded or filled within pores), and thus represent solid support media where electrophilic aromatic substitution reactions may be carried out after attachment of the proper substrate. This allows the researcher to have the ability to modify the aromatic moieties of drug precursors, or general core structures which are responsible for expression of biological activity, thereby allowing researchers access to structures which were not otherwise accessible using the existing solid phase organic chemistry tools.

Moreover, until the present invention, carbon-nitrogen bond formation dominated solid phase techniques. However, carbon-carbon bond formation, in particular, carbon-carbon bond formation in aromatic systems, is an important element in solid phase organic synthesis, especially for generating molecular diversity which may be applied toward drug lead discovery, and drug development/optimization.

In aromatic systems, carbon-carbon bond formation may be achieved by the following methods:

Friedel-Crafts alkylation with alkyl halides, alcohols, or alkenes in the presence of Lewis acids;

Friedel-Crafts acylation with acid chlorides, or acid anhydrides;

Formulation of the arylaldehyde via the Vilsmeier reaction using activated aromatic rings, dimethylformamide (“DMF”) and phosphorus oxychloride;

Michael addition; or

Transition metal mediated reactions, such as Heck reaction (palladium catalyzed C-C bond formation between aryl halide and vinyl functionality), Stille coupling (palladium catalyzed coupling of unsaturated halides or triflates with organostannanes), or Suzuki reaction (alkylboranes provide source of reactive groups).

The present invention features ceramic solid supports which are nontoxic, mechanically stable (no swelling/shrinking phenomena) and which can be used under a wide variety of reaction conditions including the reaction methods described above, and the ceramic solid supports of the present invention may be used for, e.g., organic, regular, multiple parallel, or combinatorial organic synthesis on a solid phase, or in chromatography. The solid supports of the present invention are ceramic particles having a ceramic surface material which imparts dimensional stability to the particle. By way of channels extending into the particle core material from pores in the ceramic surface, fluid (e.g., reactant or reagent in solution) contacted with the particle can permeate the material of the particle core. That is, the macrostructure of the particle is fluid permeable. In an illustrative embodiment, the particle is derived with a metal oxide ceramic surface.

In certain embodiments, the surface area material can be derivatized to support a variety of chemical functionalities. Moreover, the ceramic particles of the present invention can be made to have a selected particle size, pore size, and/or surface chemistry. The interior channels or pores of the ceramic particles, such as which may exist in, e.g., a silica core, can further be substantially filled with a three-dimensional, insoluble polymeric or gel-like material, which filling can also be employed with a variety of known chemistries The ceramic particles, with or without a pore-filling gel-like material, can also be coated with polymers, e.g., with a polymeric resin, to provide desirable surface properties.

In one aspect of the present invention, the ceramic particles are adapted for use with solid phase synthesis methods. In another aspect of the present invention, there is provided a method of using the subject ceramic particles for performing combinatorial chemistry, such as encoding tags, or using any deconvolution strategy to track compounds while avoiding any coding scheme.

Yet another aspect of the present invention concerns the use of the subject ceramic particles for chromatographic resolution, especially in columns or batches wherein the reaction conditions or apparatus impart significant forces on the particles. Still another aspect of the present invention features a method for catalytically reacting fluid streams. For this purpose, a catalytic structure is provided as a chemical functionality of the particle.

As used herein, the terms “combinatorial chemistry” or “combinatorial synthesis” include both truly combinatorial modes of synthesis and multiple parallel synthesis.

The term “particle”, as used herein, refers to a discrete unit of a material, such as a grain, a bead, or other particulate form. For most embodiments, a “particle” will not be smaller than about 1 μm, nor larger than about 10 mm. A particle may have any shape or form, although spherical or generally spherical forms are preferred.

The term “solid support particle”, as used herein, refers to a particle having a degree of three-dimensional mechanical stability. In general, the present invention features particles which are suitable for use as a solid phase in, for example, organic chemistry and peptidomimetics solid phase and combinatorial solid phase synthesis, or a variety of chromatographic formats. As described herein, the solid support may include surface reactive groups, or can be derivatized so as to contain surface reactive groups, in order to provide the particle with a reactive or binding activity with, for example, functional groups on compounds contacted with the particle, e.g., reagents, linkers, building blocks or acceptor biological molecules.

The term “pore-filled ceramic particle”, as used herein, refers to a solid support particle having interior channels or pores which are substantially filled with a three-dimensional, pore-filling gel material or polymer network derived from polymerization of effective amounts of a main monomer, a passivating or neutralizing monomer different from the main monomer, and a crosslinking agent, and optionally, a pore inducing agent and/or a polymerization initiator, as described in more detail in Girot et al., U.S. Pat. No. 5,268,097, which is incorporated herein by reference.

The terms “surface material” or “surface area”, as used herein, refer to a material that forms an exterior and an interior surface, including interior channel or pore walls, of a ceramic solid porous support particle. Preferred surface materials include, but are not limited to, ceramics comprising fused silica, zirconia, titania, alumina, ceria, hafnia, yttria, other metals, oxides, and mixtures thereof.

The term “ceramic” is art recognized and refers to a solid, refractory material produced by baking or firing of one or more essentially inorganic substances, e.g., preferably formed simultaneously, or subsequently matured, by the action of heat. Thus, ceramic includes metal oxides, carbides, borides and nitrides types, as well as carbon matrices. Exemplary ceramics include those generated from, for instance, alumina, alumina silicate, aluminium oxide, aluminium nitride, berrylia, barium titanate, fused silica, silicon carbide, silicon nitride, boron nitride, boron carbide, silicon or boron carbonitride, titanium oxide, titanium boride, titanium carbide, cerium oxide, hafnium oxide, yttrium oxide, zirconium oxide, and zirconia-toughened alumina, as well as ceramics comprising various metal oxides as a result of a firing process on a combination of two or more inorganic substances, and ceramics comprising distinct layers of various metal oxides as a result of sequential firing operations in the presence of a different inorganic substance for each firing. However, “ceramic”, as used herein, should not be unduly construed as being limited to a ceramic body in the classical sense, that is, in the sense that it consists entirely of inorganic materials, but rather refers to a body which is predominantly ceramic with respect to either composition or dominant properties.

The term “chemical functionalities” is art recognized and includes a surface that may have controlled molecular geometry and/or has surface functionality that allows various species to be attached to the surface by means of ionic, covalent, hydrogen and/or van der Waals bonding and/or molecular geometric effects, e.g., ionic exchange, affinity, frontal, size exclusion and the like. The terms “chemical functionality” or “functionality”, as used herein, are also intended to refer to a chemically reactive groups (i.e., a group capable of reacting with another chemically reactive group) such as a amine, protected amine, carbonyl, carboxyl, activated carboxyl, hydroxyl, epoxide, anhydride, thiol, carboxamido, double bond, triple bond, sulphonyl, synthones of the aforementioned functional groups, aromatic and heteroaromatic moieties, and the like.

The term “peptidomimetic moiety” is art recognized and may include, but is not limited to, unnatural amino acids or other non-peptide moieties.

The ceramic particles for use in the present invention may be made quickly and economically, adapting known methods for making ceramic materials. In addition, methods for making pore-filled ceramic particles having interior channels filled with an insoluble polymeric or gel-like material are known in the art, e.g., as disclosed in U.S. Pat. No. 5,268,097 to Girot et al.

Thus, in an illustrative embodiment, a silica (e.g., silica gel) particle is contacted with a metal salt so as to form a metal-doped silica particle. The metal-doped silica particle is then exposed to heat sufficient to ceramize the surface area of the metal-doped particle, e.g., to form a metal-oxide ceramic surface on the particle.

A metal salt will generally be selected such that the ceramic product formed upon heating has a desired quality or qualities. Suitable metals for use in forming a ceramic product include, but are not limited to, zirconium, aluminium, titanium, cerium, hafnium, calcium, magnesium, and the like. Metal salts useful for doping the ceramic include metal halides (e.g., chlorides), oxides, nitrates, sulfates, and the like, or mixtures thereof. A preferred metal is zirconium, a preferred doping agent is yttrium, and a preferred metal salt is a nitrate. A particular preferred metal salt is zirconium nitrate. A preferred process is the preparation of metal oxide on a preexisting silica porous structure. The metal salt may be brought into contact with the silica particle by, for example, dissolving or suspending the metal salt in a suitable solvent or suspending medium, and then suspending the silica particles in the metal salt mixture or spraying the metal salt mixture onto the silica particles. Alternatively, the solid metal salt may be finely divided (e.g., by grinding or milling) and intimately mixed with the silica particles before heating.

After the silica particles have been treated with the metal salt, the metal-doped silica particles are heated to in order to ceramize the particles. The optimum temperature-time ceramization program will depend on various factors, such as the ceramic precursors and the core material of the particle. In most embodiments, the heating process includes temperatures in the range of about 400° C. to about 1500° C.

In a preferred embodiment, for a zirconium nitrate-doped silica particle, firing of the particle in the ceramization process preferably occurs in the range of about 600° C. to about 1100° C., more preferably from about 700° C. to about 1100° C., and still more preferably from about 800° C. to about 1100° C.

The time of heating required will vary according to various factors, such as the size of the particles, the type of metal salt or other precursor used, and the physical and chemical characteristics of the desired ceramic surface. Furthermore, the atmosphere employed during the heating step may be selected to produce the desired characteristics of the resulting ceramic particles. For example, the pressure and composition of the atmosphere can easily be manipulated. In general, when a metal oxide-type ceramic surface is desired, it is preferable in the heating step to use an atmosphere which includes at least some oxygen, e.g., air. However, the heating step may also be performed in a vacuum, if desired. Selection of an appropriate heating time, temperature, and atmosphere conditions may be readily determined by those skilled in the art.

The size of the ceramic particle product can be controlled by selection of precursor particles of an appropriate size. For example, porous silica particles are commercially available (from, e.g., Aldrich Chemical Co., Milwaukee, Wis.) in a variety of sizes and porosities which can be selected to determine the size and porosity of the ceramic particle product. For example, silica gel particles are available in sizes from about 5 μm to about 3 mm, and with average pore sizes in the range of about 40 angstroms to about 6000 angstroms. Thus, the size of the ceramic particle product is generally in the range of about 5 μm to about 3 mm, and more preferably about 10 μm to about 1 mm. Particles suitable for use as solid supports for liquid chromatography or continuous flow solid phase synthesis preferably have a diameter in the range of about 10 μm to about 1 mm, and more preferably about 50 μm to about 500 μm.

The skilled artisan will appreciate that the ceramic particles of the present invention may be separated by size according to methods known in the art, making available a desired size or range of sizes. For example, the ceramic particles can be sieved or otherwise screened to remove particles above or below a preselected size or range.

For use as solid supports for solid phase and combinatorial solid phase synthesis, wherein the ceramic surface material is derivatized or functionalized to support an immobilized molecule, it is preferable to employ a ceramic support which has a pore size large enough to allow access of reagents and solvents to the interior of the particle. Thus, in preferred embodiments, a porous ceramic solid support of the present invention has a mean pore sized of at least 100 angstroms , and more preferably at least 1500 angstroms.

In certain embodiments of the present invention, it is preferred that at least some of the pores that permeate the interior of the particle are filled with an insoluble polymeric material. Exemplary insoluble polymeric materials that can be employed include polymer resins such as are conventionally employed for solid phase synthesis, such as polystyrene resins, polydimethylacrylamide gels, and the like, or suitable functionalized derivatives thereof. In embodiments of the present invention pore-filled ceramic particles, wherein interior pores of a ceramic particle are filled with an insoluble polymeric material or polymer network, the pore size of the ceramic particle is preferably in the range of about 500 angstroms to about 3000 angstroms, more preferably from about 800 to about 1500 angstroms. The pores of the ceramic particles may be filled after ceramization of the exterior surface of the particles, for example, by suspending the ceramic particles in a solution or suspension of at least one monomer, allowing the monomer (or monomers if a copolymer is desired) to infiltrate the pores of the ceramic particles, and initiating polymerization. Suitable conditions for filling the pores of a particle with a polymeric resin are described in, e.g., U.S. Pat. No. 5,268,097 to Girot et al. It will be appreciated by those skilled in the art that some processes may result in coating of the particles with the polymer. This coating can be removed, if desired, by methods known in the art.

In embodiments of the present invention in which interior pores are not filled with a polymeric resin, all the surface area of the ceramic particles, including interior channel or pore surfaces, may be chemically derivatized to provide desirable properties. For example, surfaces can be derivatized with silane compounds to provide a variety of surface chemistries. Techniques for such derivitizations are well known by the skilled artisan, and allow the introduction of functionalities such as amines, carboxylates, alkyl halides, aromatic moieties, heteroaromatic moieties, aminosilanes, phenyltrichloromethyl silane, & arenediazonium salts and the like. Thus, well-known chemistries for the attachment of an organic molecule to a solid support may be used to bind the ceramic particles of the present invention to an organic molecule for solid phase or combinatorial solid phase synthesis.

The ceramic particles of the present invention advantageously have high loading capacities for organic compounds. High loading capacity is the result of the large surface area of the ceramic material when the surface is used to attach organic molecules or coating polymers. With pore-filled ceramic particles, high loading capacity is the result of the three-dimensional structure of the pore-filling polymer network. In other words, the ceramic particles of the present invention feature high densities of reactive sites. In preferred embodiments, the ceramic particles of the present invention provide, in terms of mequiv of reactive sites per gram of ceramic particles, at least about 0.1 mequiv/g, preferably at least about 0.3 mequiv/g, more preferably at least about 0.5 mequiv/g, even more preferably at least about 1.0 mequiv/g, and still more preferably at least about 1.5 mequiv/g. Thus, for example, ceramic particles of the present invention, when derivatized with an alkyl amino functionality, will in a more preferred embodiment have at least about 1.5 mequiv of amino functionalities per gram of ceramic particles. Particles with high loading capacities are suitable for synthesis of larger amounts of organic compounds than are particles with lower loading capacities.

The surface material may also be derivatized according to known methods. In addition, the ceramic particles of the present invention may be derivatized with one or more reagents, such that various portions of the particle may have the same or different functional groups. For example, if the interior pores of the particle are filled with a polymeric gel carrying carboxy groups, derivatization of the ceramic surface (exterior) can be performed such that, for instance, this surface has amino functionalities. Polymer and ceramic surfaces may also be independently functionalized, if desired, by temporarily preventing a surface from reacting with a derivatizing agent. In practice, the interior pores can be filled, e.g., as described above, and the ceramic surface then derivatized. For example, if the interior pores are filled with a fugitive additive, the ceramic surface can be derivatized, and then the fugitive additive driven off.

Furthermore, the ceramic surface can be selected such that the surface has catalytic properties for preselected reactions. For example, a zirconium oxide ceramic surface can function as a catalyst for certain types of reactions (for example, as a Lewis acid in acid-catalyzed reactions).

The ceramic particles of the present invention may be used as solid supports for solid phase synthesis and combinatorial solid phase synthesis according to methods known in the art. Thus, the mineral surface of the ceramic particles, or the polymer network of pore-filled ceramic particles, can be derivatized with functional groups which permit the attachment of an organic molecule to the surface, and the organic molecule can then be subjected to reactions which result in synthesis of desired molecules. For example, for solid phase peptide synthesis, the ceramic surface can be treated with a reagent such as an aminosilane to produce an amino-derivatized ceramic surface (including interior channel or pore surfaces), or alternatively, using pore-filled ceramic particles the components of the polymer network can be selected such that the polymerized product contains primary amino groups. An amino acid can then be coupled to the amino functionality by conventional methods, and the polypeptide chain elongated by conventional solid phase peptide synthesis Techniques. Optionally, an appropriate or compatible linker may first be attached to the amino functionality in order to facilitate cleavage of the resulting polypeptide chain.

Similarly, the ceramic surface material, or the three-dimensional polymer networks of pore-filled ceramic particles, may be derivatized to yield a phenyl-functionalized ceramic surface material or polymer network. The phenyl groups may then be derivatized to yield an appropriate functionality or reaction group, which may be modified and used for attachment of a compatible or appropriate linker, and peptide synthesis can then proceed according to known methods (see, e.g., M. Bodansky, “Principles of Peptide Synthesis”, 2nd Ed, Springer-Verlag (1993)). When peptide synthesis is complete, the product polypeptide can be cleaved from the ceramic particle (see, e.g., G. Barany and R. B. Merrifield, in E. Gross, J. Meinhofer, eds., “The Peptides”, Vol. 2, Academic Press, New York (1979)) and purified to provide the desired product.

In analogous fashion, the ceramic particles of the present invention may be used for the synthesis of other molecules, including, but not limited to, other polymeric molecules, such as nucleic acids or oligosaccharides, non-polymeric molecules, small drug like molecules, and other such drug like molecules according to known methods.

The subject ceramic and pore-fiiled ceramic particles can be advantageously employed under conditions wherein many conventional solid phase supports are not stable or are unsuitable. For example, the ceramic and pore-filled ceramic particles of the present invention have sufficient mechanical strength to be employed under conditions of moderate to high pressure In addition, the ceramic and pore-filled ceramic particles of the present invention are relatively inert in comparison with polymeric resin supports, and thus may be used under vigorous reaction conditions which cannot be used with conventional supports. Furthermore, the ceramic and pore-filled ceramic particles of the present invention do not substantial. swell or shrink in most solvents, and so are compatible with a wide range of organic solvents. The ceramic and pore-filled ceramic particles of the present invention also have a relatively low coefficient of expansion, and thus do not significantly swell or shrink at high or low temperatures. For example, the particles of the present invention can generally be used at temperatures between about −80° C. and about 150° C. without significant degradation of the particles.

Ceramic and pore-filled ceramic particles of the present invention have higher specific density than most common organic solvents used in organic synthesis. Thus, the particles of the present invention are easy to handle during separation processes, such as decantation, and during particle redistribution when performing combinatorial mode of organic synthesis either manually or in automated fashion.

According to the present invention, aromatic and heteroaromatic moieties, such as phenyl, biphenyl, naphthyl, indolyl and the like, may be attached to ceramic particles (either on the ceramic surface or on the three-dimensional polymer network of pore-filled ceramic particles) which are then used as solid supports for solid phase synthesis and solid phase combinatorial chemistry methods which are incompatible with existing supports. For example, Friedel-Crafts acylation and alkylation may be used to derivatize aromatic moieties attached to the ceramic particles. The solid support particles are not affected by the conditions needed for Friedel-Crafts reactions. However, solid supports prior to the present invention excluded these reactions from the reaction manifold of organic chemists because of incompatibility with the existing resin supports themselves, although Friedel-Crafts reaction was used to functionalized a polystyrene polymer to introduce chloromethyl, aminomethyl and other groups (Barany G., Merrifield R. B. in “The Peptides (E. Gross and J. Meienhofer, eds.), vol. 2, Academic Press, New York, vol. 2, pp. 1-284 (1979); Stewart, J. M., Young, J. D. in “Solid Phase Peptide Synthesis”, 2nd edition, Pierce Chemical Co., Rockford, Ill.(1984)).

Hence, the ceramic particles and pore-filled ceramic particles of the present invention may be used as solid supports for combinatorial syntheses which utilize Friedel-Crafts reaction as a means of modifying the moieties attached to the ceramic particles. This is an important feature of the present invention, as it allows one to generate molecularly diverse compounds by modifying aromatic moieties on a solid support without modification of the solid support matrix itself. In particular, Friedel-Crafts acylation and alkylation reactions permit derivatization of benzene rings attached to the ceramic particle, which is very useful as a majority of known drugs contain a benzene ring.

In addition, the ability to use Friedel-Crafts reactions with the ceramic particles or porous ceramic solid supports of the present invention allows one to use combinatorial chemistry for research and/or generation of linkers, especially photocleavable linkers. Linkers are usually prepared separately using solution phase chemistry, then they are attached to a functionalized resin.

The ceramic particles of the present invention allow generation of combinatorial libraries of photocleavable and/or other linkers, such as, but not limited to, benzhydryl or diphenylmethane based linkers, which linkers may be subsequently tested in a combinatorial mode.

For example, linker-ceramic particles may be synthesized according to reaction Scheme (I) set forth below:

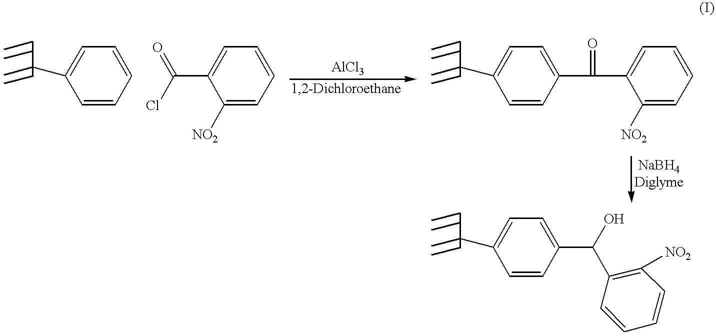

wherein the four lines represent the ceramic particle.

The first step of reaction Scheme (I) involves Friedel-Crafts reaction of aromatic compound-particles with o-nitrobenzoyl chloride in the presence of anhydrous aluminum chloride. Reduction of the resulting ketone-particles with sodium borohydride in diglyme affords 2′-Nitrobenzhydryl linker-ceramic particles.

Moreover, the ceramic particles of the present invention may be used for solid phase synthesis of desired linkers. These linkers can then be cleaved from the particles and used where they are needed. Such an approach does not limit the researcher to using only one particular solid support, but rather allows the researcher to attach such prepared linkers to different solid supports, e.g., various resins such as polystyrene based resins, TentaGel (commercially available from Rapp Polymere Gmbh, Tubingen, Germany), ceramic particles of various compositions, ceramic particles with various coatings, and the like. Thus, the present invention provides a high level of orthogonality control.

The ceramic particles of the present invention may also be subjected to electrophilic aromatic/heteroaromatic substitutions, and the resulting functionalized ceramic particles may be used as solid supports for subsequent solid phase combinatorial syntheses. For example, chlorobenzotrifluorides react under typical Friedel-Crafts reaction conditions with aromatic compounds to afford 1,1-Dichlorodiphenylmethanes. Thus, according to the present invention, ceramic particles functionalized with an aromatic moiety may be reacted with ArCF₃ under Friedel-Crafts alkylation conditions as an avenue to perchloro compounds, as set forth below in reaction Scheme (II):

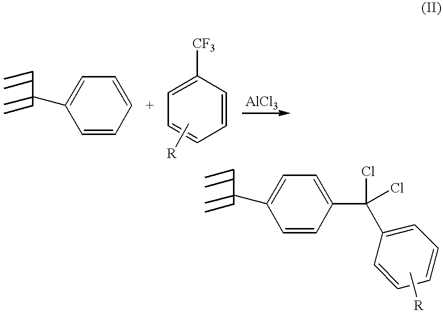

wherein the four lines represent the ceramic particle, and R represents any desired substituent, including, but not limited to, an aliphatic group, such as a C₁-C₂₀ aliphatic group, an alicyclic group, aromatic moieties, and heteroaromatic moieties.

This scheme allows for the generation of ArC⁺Cl₂ alkylates and arenes, as well as thiophene. (Ramchandani Roshan K., Wakharbar R. D., Sudalai A., Tetrahedron Letters, 37(23): 4063 (1996)). Perchloro compounds have received extensive interest in pharmaceutical and agrochemical industry, and thus the ability to rapidly synthesize a library of perchloro compounds using solid phase combinatorial chemistry is extremely advantageous.

The ceramic particles of the present invention may also be used in conjunction with reversible Friedel-Crafts acylation of pyrrole, and the resulting functionalized ceramic particles used as solid support particles for solid phase combinatorial synthesis. For example, while pyrroles normally undergo substitution at the C-2 position, it has been shown that 1-(phenyisulfonyl)pyrrole displays a tunable reactivity in Friedel-Crafts acylation. (Xu et al., Tetrahedron Letters, 22:4899 (1981); Rokach et al, Tetrahedron Letters, 22:4901 (1981); Kakushima et al., J. Org. Chem., 48:3214 (1983)). The differential reactivity is largely controlled by the acid catalyst used. Ketcha et al. described Friedel-Crafts acylation of 3-alkyl-1-(phenylsulfonyl)pyrroles. (Ketcha et al., Tetrahedron Letters, 37:1523 (1996)).

This reaction appears to be kinetically favored at the C-2 position of the pyrrole ring, but rearrangement to the C-5 position can occur after prolonged reaction times. As a result, these reaction schemes may be used to provide access to 3- and 5-substituted pyrroles, which may be synthesized using the ceramic particles of the present invention, as set forth below in reaction Scheme (III):

[chemical structure]

wherein the circle represents the ceramic particle, and R represents any desired substituent, including, but not limited to, an aliphatic group, such as a C₁-C₂₀ aliphatic group, an alicyclic group, aromatic moieties, and heteroaromatic moieties. The broken lines in reaction Scheme (III) indicate that the pyrrole structure may be attached directly to a linker or handle, and/or may be attached to or be a part of a more complex structure.

The various reaction schemes discussed above provide examples of synthetic avenues which may be used to create aromatic and heteroaromatic series of combinatorial libraries when, according to the present invention, ceramic particles are used as solid supports for solid phase syntheses. The molecular diversity which may be achieved as a result of using ceramic particles according to the present invention, which particles are not affected during electrophilic aromatic substitutions, as the solid support in solid phase combinatorial syntheses represents a very important advance for drug discovery and drug development. In particular, the present invention allows researchers to have access to a variety of heterocyclic structures which play very important roles in drug design.

The present invention also provides methods for combinatorial synthesis of organic compounds using the ceramic particles of the present invention as solid supports. The synthesis of combinatorial libraries is well known in the art (see, e.g., Gordon etal., J. Med. Chem., 37:1385-1401 (1994); and Gallop, M. A., J. Med. Chem, 37:1233-1251 (1994)). For example, libraries of small organic molecules, such as benzodiazepines, have been synthesized on solid supports (see Bunin et al., Proc. Natl Acad. Sci. USA, 91:4708-4712 (1994); DeWitt et al., Proc. Nat'l Acad. Sci. USA, 90:6909-6913 (1993); and Bunin, B. A. and Ellman, J. A., J. Am. Chem. Soc., 114:10997-10998 (1992)). Such libraries can be synthesized according to a variety of methods. For example, a “diversomer library” can be created according to the method of Hobb DeWitt et al. (Proc. Nat'l Acad. Sci. U.S.A., 90:6909-6913 (1993)), using the ceramic particles of the present invention in place of conventional resin supports. Aliquots of the ceramic particles of the present invention, preferably having either interior surfaces, or the three-dimensional polymer network in pore-filled ceramic particles, functionalized to permit linking of an organic moiety to the ceramic particle, are placed in an array of reaction vessels, and one of a plurality of reactants is introduced into each vessel. The reactants are allowed to react with the functionalized ceramic material. After reaction, the organic moieties are linked to the ceramic particles. The particles are washed to remove impurities, reagents and excess reactants, and the particles are divided, if desired, into further reaction vessels. The immobilized organic moieties are then subject to further reactions, washed, and cleaved from the ceramic particles to yield the desired library of organic compounds.

In another embodiment, a library of organic compounds may be synthesized on the ceramic particles of the present invention according to the “split-pool” method of combinatorial synthesis (see, e.g., Lam et al., Nature, 354:82-84 (1991)). In this embodiment, the ceramic particles are first functionalized as previously described, and then divided into a plurality of reaction vessels. Each aliquot of particles is treated with a different reagent. When reaction is complete, the aliquots of ceramic particles (with organic moieties linked thereto) are recombined. The combined pool of ceramic particles is then again divided into a plurality of reaction vessels, and each aliquot is exposed to a different reagent. The cycle of dividing, reacting, and recombining, is repeated until the desired library of compounds is formed. This technique is especially useful in combination with the encoding methods described below.

Other synthesis methods, including the “tea bag” technique of Houghten (see Houghten etal., Nature, 354:84-86 (1991)) may also be used to synthesize libraries of compounds according to the present invention.

Combinatorial libraries may be screened to determine whether any members of the library have a desired activity, and, if so, to identify the active species. Some methods of screening combinatorial libraries have been described (see, e.g., Gordon et al., J. Med. Chem., op. cit.). Soluble compound libraries can be screened by affinity chromatography with an appropriate receptor to isolate ligands of the receptor, followed by identification of the isolated ligands by conventional techniques, such as mass spectrometry, NMR, IR, UV, and the like. Immobilized compounds can be screened by contacting the compounds with a soluble receptor; preferably, the soluble receptor is conjugated to a label (e.g., fluorophores, calorimetric enzymes, radioisotopes, luminescent compounds, and the like) that can be detected to indicate ligand binding. Alternatively, immobilized compounds can be selectively released and allowed to diffuse through a membrane to interact with a receptor.

Using the ceramic particles of the present invention as solid supports, combinatorial libraries of compounds may also be synthesized with “tags” to encode the identity of each member of the library (see, e.g., W. C. Still et al., PCT Publication No. WO94/08051; and Zuckermann et al, PCT Publication No. WO94/13623). In general, this method features the use of inert, but readily detectable, tags that are attached to the substrate compound. When an active compound is detected (e.g., by one of the screening techniques set forth above), the identity of the compound is determined by identification of the unique accompanying tag. This tagging method permits the synthesis of large libraries of compounds which can be identified at very low levels. The ceramic particles of the present invention can be used as solid supports in encoded synthesis schemes according to conventional techniques. The particles of the present invention also permit the use of topologically segregated encoding schemes, such as are described in Lebl et al. (PCT Publication No. WO94/28028). For example, the organic polymer coat of ceramic material, or the three-dimensional polymer network of pore-filled ceramic particles, can be derivatized to permit the synthesis of the combinatorial library, while the ceramic surface is used for coding information.

For example, the libraries of compounds synthesized according to the present invention may contain at least forty-eight (48) compounds, more preferably at least ninety-six (96) compounds. In preferred embodiments, the libraries of compounds contain up to about 10⁷ compounds, more preferably up to about 10⁶ compounds, and still more preferably, up to about 10⁵ compounds.

The invention is further illustrated by reference to the following examples. It will be apparent to those of skill in the art that many modifications, both to materials and methods, may be practiced without departing from the purpose and scope of this invention.

### EXAMPLE 1

### Preparation of Silica Ceramic Particles With Zirconium Oxide Coating

50 g of porous silica (average particle size 50 μm; pore volume 1 ml/fg; pore size 2500 Angstrom) were washed extensively with 6 M hydrochloric acid so as to eliminate sodium hydroxide ions that may be present. This porous silica was then rinsed with distilled water until neutral and dried. 50 ml of a solution of zirconyl oxynitrate at a concentration of 105 g/liter in 1 M nitric acid was prepared. The mixture was stirred until a clear acidic solution was obtained.

This solution and the dry silica powder were mixed together by slowly adding the liquid solution at room temperature to the silica powder while agitating the powder inside a closed vessel. Once the addition of the solution was completed, agitation of the mixture was continued for about 30 to 60 minutes.

The resulting acidic paste was placed inside an oven. The temperature of the oven was first brought to about 100-150° C. for about an hour, and then increased progressively up to about 975-1000° C. The resulting solid mineral material was then maintained at this temperature for three hours, and then progressively cooled overnight to room temperature.

The resulting dry ceramic material was suspended in about 120 ml of distilled water under gentle agitation for about 15-30 minutes, at which time agitation was stopped and the supernatant eliminated by decantation. This procedure was then repeated 3-4 times until a clear supernatant was obtained. Hydrochloric acid was then added under agitation at a final concentration of 1 M (two volumes of decanted material). Ceramic particles were maintained in suspension under agitation for about one hour.

The solution was neutralized and the particles washed extensively with distilled water. All the water was drained by vacuum filtration and the resulting cake dried under vacuum at about 85° C. for 16-24 hours.

The material obtained was composed of silica ceramized with a coat of zirconium oxide. This material was very stable in alkaline media, while the properties of porosity and particle size were essentially unchanged compared to the porous silica starting material.

### EXAMPLE 2

### Preparation of Ceramic Particles With a Mixture of Metal Oxides

100 g of porous silica (average particle size 70 μm; pore volume 1 ml/g; pore size 1250 Angstrom) were washed extensively with 6 M nitric acid so as to eliminate sodium ions that may be present. This porous silica was then rinsed with distilled water until neutral and dried. 100 ml of a solution of zirconyl nitrate, aluminum nitrate, and calcium nitrate, at concentrations of 40 g/liter, 40 g/liter, and 20 g/liter in 1 M nitric acid, respectively, was prepared. The mixture was stirred until a clear acidic solution was obtained.

This solution and the dry silica powder were mixed together by slowly adding the liquid solution at room temperature on the silica powder inside a closed vessel. Once the addition of the solution was completed, agitation of the mixture was continued for about 30 to 60 minutes.

The resulting acidic paste was placed inside an oven. The temperature of the oven was first brought to about 100-150° C. for about an hour, and then increased progressively up to about 975-1000° C. The resulting mineral solid material was then maintained at this temperature for three hours, and then progressively cooled overnight to room temperature.

The resulting dry ceramic material was then suspended in about 120 ml of distilled water under gentle agitation for about 15-30 minutes, at which time agitation was stopped and the supernatant eliminated by decantation. This procedure was then repeated 3-4 times until a clear supernatant was obtained. Acetic acid was then added under agitation at a final concentration of 1 M (two volumes of decanted material). Ceramic particles were maintained in suspension under agitation for about one hour. The solution was neutralized and the particles washed extensively with distilled water. All the water was drained by vacuum filtration and the resulting cake dried under vacuum at about 85° C. for 16-24 hours.

The material obtained was composed of silica ceramized with a coat of zirconium oxide, aluminum oxide and calcium oxide. This material was very stable in alkaline media, while the properties of porosity and particle size were essentially unchanged compared to the porous silica starting material.

### EXAMPLE 3

### Preparation of Ceramic Particles With Catalytic Properties

Preparation of ceramic particles was done in a similar manner to Examples 1 and 2 above, except that the solution of mineral salts contained 10 g/liter of platinum nitrate. The resulting particles have the same physical properties as regular ceramized silica particles (e.g., high mechanical stability, alkaline stability, and stability at any temperature below about 800° C. in any common organic solvent), except that these particles also possess catalytic properties for certain applications due to the presence of platinum oxide.

### EXAMPLE 4

### Surface Coating of Ceramic Particles With a Film of a Functional Polymer Containing Amino Groups

60 ml of ethanol 95% were mixed under agitation with 4 ml of polyethyleneimine (mother solution was 50% in water). The mixture was made at room temperature (15-25° C.). After a few minutes, 36 ml of distilled water and 1.2 ml of butanedioldiglycidylether were added, and the resulting mixture was stirred until homogeneous clear solution was obtained.

This solution was then mixed with 100 g of the ceramic material obtained in Example 1 above, and agitated extensively so as to obtain an apparent dry powder (however, the pores are filled with the polyethyleneimine solution). The resulting material was transferred into a closed vessel, and heated moderately at about 80-85° C. for about three (3) hours. Evaporation of the solvent (water and ethanol) was performed under vacuum, and the dry material was then washed extensively with distilled water to eliminate by-products and unreacted organic material.

The resulting product showed the presence of amino groups on the surface of the ceramic material, wherein the amount of amino groups was about 0.24 mequiv per dry gram of ceramic.

### EXAMPLE 5

### Pore Filling of Ceramic Particles With a Carboxyl-Containing Functionalized Polymer Gel

1.2 g of N,N′-methylene-bis-methacrylamide was dissolved in 50 ml of dimethylsulfoxide, and then 22 ml of acrylic acid was added under stirring. 0.32 g of azobis amidino propane was added and the total volume of the solution was adjusted to 85-95 ml by adding demineralized water. The final volume depends on the pre-volume of the dry ceramic particles. The pH of the solution was checked, and adjusted to between 3 and 5 with the addition of sodium hydroxide.

The resulting solution was mixed with 100 g of dry silica ceramic particles (surface area of about 20 cm²/g , pore volume of about 0.9-1.1 ml/g, and pore size of about 2500 Angstrom), and stirred thoroughly in order to have the total solution adsorbed by capillary action into the pore volume. The mixture was transferred into a closed vessel and heated at 85-90° C. for four hours. The mixture was then slowly cooled to room temperature overnight.

The resulting polymer, inside the ceramic material, was resuspended in a large excess of deionized water, and gently agitated. Agitation was stopped after about one hour, and the supernatant was eliminated by decantation. Several of these washing were done until a clear supernatant was obtained. The particles were then treated with one volume of 1 M sodium hydroxide for 30 to 45 minutes. The supernatant was removed and the gel was extensively washed with deionized water until a neutral pH, and then dried at a temperature of 80-85° C.

The resulting ceramic particles carried free carboxyl groups in an amount of about 0.8 mequiv per gram of dry ceramic material. Such functional groups are available for subsequent chemical reactions for solid phase synthesis.

### EXAMPLE 6

### Pore Filling of Ceramic Particles With a Carboxyl-Containing Functionalized Polymer Gel

1.2 g of N,N′-methylene-bis-methacrylamide was dissolved in 50 ml of dimethylsulfoxide, and then 22 ml of acrylic acid was added under stirring. 0.32 g of azobis amidino propane was added and the total volume of the solution was adjusted to 85-95 ml by adding demineralized water.

The final volume depends on the pre-volume of the dry ceramic particles. The pH of the solution was checked, and adjusted to between 3 and 5 with the addition of sodium hydroxide.

The resulting solution was mixed with 100 g of dry zirconia particles (surface area of about 25 cm²/g , particle diameter of about 50 μm, and pore diameter of about 1500 Angstrom), and stirred thoroughly in order to have the total solution adsorbed by capillary action into the pore volume. The mixture was transferred into a closed vessel and heated at 85-90° C. for four hours. The mixture was then slowly cooled to room temperature overnight.

The resulting polymer, inside the ceramic material, was resuspended in a large excess of deionized water, and gently agitated. Agitation was stopped after about one hour, and the supernatant was eliminated by decantation. Several of these washing were done until a clear supernatant was obtained. The particles were then treated with one volume of 1 M sodium hydroxide for 30 to 45 minutes. The supernatant was removed and the gel was extensively washed with deionized water until a neutral pH, and then dried at a temperature of 80-85° C.

The resulting ceramic particles carried free carboxyl groups in an amount of about 0.4 mequiv per gram of dry ceramic material. Such functional groups are available for subsequent chemical reactions for solid phase synthesis.

### EXAMPLE 7

### Surface Area Grafting of Ceramic Particles With Primary Amino-Containing Ligands

50 g of well-dried ceramic particles (particle size of about 70 μm, surface area of about 200 m²/g, and pore diameter of about 300 Angstrom) were mixed with 100 ml of a mixture of amino-propyl-3-ethoxy-silane and toluene (50:50). The suspension was boiled overnight under reflux. The resulting grafted particles were collected by filtration, washed extensively with the non-polar solvents toluene, and then acetone, followed by washing with ethanol, and then dried under vacuum at about 85° C. for 16-24 hours.

The resulting ceramic grafted particles showed available primary amino groups for subsequent chemical reaction for solid phase synthesis. The amount of amino groups was about 0.4 mequiv per gram of dry ceramic material.

### EXAMPLE 8

### Surface Area Modification of Ceramic Particles With Tentacle-Like or Growing Functionalized Monomers

Silica ceramic particles with primary amino functionality, as described in Example 7 above, were reacted with methyl methacrylate to introduce two molecules of this compound with two double acrylic bonds available for subsequent reactions. That is, two chemical functions (two acrylic double bonds) were obtained from one chemical function (a primary amino group).

The resulting intermediate product having double bonds was reacted with ethylenediamine to give rise to another intermediate solid phase carrying primary amino groups. If desired, the number of amino groups may be expanded by factors of two by running another reaction(s) as described above, i.e., by first reacting the intermediate particles with methylmethacrylate molecules followed by ethylenediamine. Such an expanding reaction is well known in the art for the preparation of dendrimers in solution.

Once the desired number of primary amino groups is obtained, the resulting ceramic particles may be used as a solid support to start appropriate solid phase synthesis or combinatorial synthesis.

### EXAMPLE 9

### Derivatization of Aromatic Compound Functionalized Ceramic Particles

Introduction of phthalimide protected aminoethyl group:

Ceramic particles functionalized with an aromatic compound (compound-particle (analogous to compound-resin used in solid phase chemistry)) are placed into a reactor (fritted glassware) with nitrogen bubbling. N-(chloromethyl)-phthalimide (about 2 fold molar excess with respect to the particle loading) and dichloromethane (minimum amount needed for dissolution) are placed into the reactor. The particles are suspended by bubbling, and 0.3-0.4 mol of catalyst per mol of substrate) AlCl₃ or FeCl₃ is added. After 2 hours of bubbling, the particles are washed with dioxane, 1 N HCl-dioxane (1:1), methanol and dried under vacuum.

Deprotection:

The resulting phthalimidomethyl compound-particles are refluxed with 5% hydrazine hydrate in ethanol yielding aminomethyl compound-particles. Free amino groups can be quantitatively determined using well-known procedures for quantitation of amino groups on solid supports, e.g., picric acid test (J. M. Stewart and J. D. Young in “Solid Phase Peptide Synthesis”, p. 107, 2nd edition, Pierce Chemical Co., Rockford, Ill. (1984)), or spectrophotometric determination of cleaved phthalimide groups, or Fmoc UV reading at 302 nm.

### EXAMPLE 10

### Friedel-Crafts Reaction of Benzene-Ceramic Particles To Synthesize 2′-Nitrobenzhydryl linker-Ceramic Particles

Ceramic particles functionalized with a benzene ring are placed in a round-bottom flask and 1,2-dichloroethane is added. About 3 fold molar excess of o-nitrobenzoyl chloride is added, followed by slow addition of 4 fold molar excess of anhydrous aluminum chloride. The reaction mixture is refluxed for about 16 hours. Dioxane:4N-HCl (3:1) is added to the reaction mixture and allowed to cool in an ice bath. The resulting ketone-particles are filtered using a course glass frit, washed with dioxane-4N-HCl (3:1), dioxane-water, dioxane, methanol and dichloromethane stepwise (alternatively, a continuous flow method may be used), and then dried under vacuum.

### Reduction of ketone-particles

The resulting dried ketone-particles are bubbled with nitrogen in diglyme. A solution of sodium borohydride (2 M) in diglyme is added at 0° C. dropwise over 30 minutes, and then bubbled at 55° C. for 24 hours. The reaction mixture is cooled to 0° C. and 2N HCl is added slowly The resulting 2′-Nitrobenzhydryl linker-ceramic particles are thoroughly washed with hot water, methanol and dichloromethane, and dried under vacuum.

### EXAMPLE 11

### Friedel-Crafts Reaction of Benzene-Ceramic Particles To Synthesize 1,1-Dichlorodiphenylmethanes-Ceramic Particles

4-Chlorobenzotrifluoride (1 equivalent) is added to a stirred/bubbled solution of cooled (0° C.) anhydrous AlCl₃ (3 equivalents) in 1,2-dichloro-ethane upon which a red-colored complex is formed. Benzene-ceramic particles corresponding to 1 equivalent are added to this complex all at once (or vice versa), and the reaction mixture is bubbled for 3 hours at 0° C. The resulting 1,1-di-chlorodiphenylmethane-ceramic particles are filtered, thoroughly washed with water, DMF, methanol, and dichloromethane, and then dried under vacuum.

The embodiments of the present invention described above are intended to be merely exemplary and those skilled in the art will recognize, or be able to ascertain using no more than routine experimentation, numerous equivalents to the specific procedures described herein. All such equivalents are considered to be within the scope of the present invention and are covered by the following claims.

The contents of all references described herein are hereby incorporated by reference.

Other embodiments are within the following claims.

What is claimed is:

1. A porous ceramic solid support for use in solid phase synthesis of molecules comprising ceramic particles, wherein said ceramic particles comprise a ceramic surface material having exterior and interior surfaces, and pores which permeate the interior of the ceramic particles, and wherein said ceramic surface material is derivatized with one or more chemical functionalities.

2. A porous ceramic solid support for use in solid phase synthesis of molecules comprising pore-filled ceramic particles, wherein said pore-filled ceramic particles comprise a ceramic surface material having exterior and interior surfaces, and pores which permeate the interior of the ceramic particles which are substantially filled with a three-dimensional polymer network, and wherein said three-dimensional polymer network is derivatized with one or more chemical functionalities.

3. The porous ceramic solid support of claim 1 or claim 2, wherein said ceramic surface material is coated with a polymer to provide desired surface properties.

4. The porous ceramic solid support of claim 1 or claim 2, wherein the ceramic surface material is selected from the group consisting of alumina, alumina silicate, aluminium oxide, aluminium nitride, berrylia, barium titanate, fused silica, silicon carbide, silicon nitride, boron nitride, boron carbide, silicon or boron carbonitride, titanium, titanium oxide, titanium boride, titanium carbide, hafnium, hafnium oxide, cerium, cerium oxide, zirconium, zirconium oxide, yttrium, yttrium oxide, zirconia-toughened alumina, and mixtures thereof.

5. The porous ceramic solid support of claim 4, wherein the ceramic surface material is a mixture selected from the group consisting of zirconium oxide and cerium oxide, zirconium oxide and hafnium oxide, and zirconium oxide and yttrium oxide.

6. The porous ceramic solid support of any one of claims 1, 2, or 4, wherein said chemical functionalities are selected from the group consisting of aromatic moieties, heteroaromatic moieties, amine protected amine, carbonyl, carboxyl, activated carboxyl, hydroxyl, epoxide, anhydride, thiol, and carboxamido.

7. The porous ceramic solid support of claim 1 or claim 2, wherein said porous ceramic solid support provides at least about 0.1 mequiv of reactive sites per gram of ceramic particles.

8. The porous ceramic solid support of claim 7, wherein said porous ceramic solid support provides at least about 0.3 mequiv of reactive sites per gram of ceramic particles.

9. The porous ceramic solid support of claim 8, wherein said porous ceramic solid support provides at least about 0.5 mequiv of reactive sites per gram of ceramic particles.

10. The porous ceramic solid support of claim 9, wherein said porous ceramic solid support provides at least about 1.0 mequiv of reactive sites per gram of ceramic particles.

11. The porous ceramic solid support of claim 1 or claim 2, wherein the ceramic particles have an initial average particle size ranging from about 5 μm to about 3 mm.

12. The porous ceramic solid support of claim 11, wherein the initial average particle size is from about 10 μm to about 1 mm.

13. The porous ceramic solid support of claim 1 or claim 2, wherein the ceramic particles have an initial pore size ranging from about 40 Angstroms to about 6000 Angstroms.

14. The porous ceramic solid support of claim 2, wherein the pore-filled ceramic particles have an initial pore size ranging from about 500 Angstroms to about 3000 Angstroms.

15. The porous ceramic solid support of claim 14, wherein the initial pore size is from about 800 Angstroms to about 1500 Angstroms.

16. A method for solid phase synthesis of molecules using a porous ceramic solid support comprising the steps of:
(a) derivatizing ceramic surface material of said porous ceramic solid support with one or more chemical functionalities which permit attachment of an organic molecule to said ceramic surface material;
(b) attaching said organic molecule to the ceramic surface material; and
(c) subjecting the organic molecule to reactions which result in synthesis of said molecules.

17. A method for solid phase synthesis of molecules using a porous ceramic solid support, wherein said porous ceramic solid support comprises pore-filled ceramic particles, comprising the steps of:
(a) derivatizing a three-dimensional polymer network within interior channels of said pore-filled ceramic particles with one or more chemical functionalities which permit attachment of an organic molecule to said three-dimensional polymer network;
(b) attaching said organic molecule to the three-dimensional polymer network; and
(c) subjecting the organic molecule to reactions which result in synthesis of said molecules.

18. The method for solid phase synthesis of molecules of claim 16, wherein the ceramic surface material is selected from the group consisting of alumina, alumina silicate, aluminium oxide, aluminium nitride, berrylia, barium titanate, fused silica, silicon carbide, silicon nitride, boron nitride, boron carbide, silicon or boron carbonitride, titanium, titanium oxide, titanium boride, titanium carbide, hafnium, hafnium oxide, cerium, cerium oxide, zirconium, zirconium oxide, yttrium, yttrium oxide, zirconia-toughened alumina, and mixtures thereof.

19. The method for solid phase synthesis of molecules of claim 16 or 17, wherein said molecules are selected from the group consisting of nucleic acids, oligosaccharides, polypeptides, and peptidomimetics.

20. The method for solid phase synthesis of molecules of claim 16 or 17, wherein said molecules non-polymeric molecules selected from the group consisting of aromatic and heteroaromatic molecules, small molecules, and other drug-like molecules.

21. A method for solid phase synthesis of polypeptides or peptidomimetics using a porous ceramic solid support comprising the steps of:
(a) derivatizing ceramic surface material or a three-dimensional polymer network of said porous ceramic solid support with an amino functionality;
(b) attaching a linker compatible with used chemistry to said amino functionality and coupling a first amino acid or peptidomimetic moiety to said linker;
(c) coupling one or more additional amino acids or peptidomimetic moieties to said first amino acid or peptidomimetic moiety;
(d) cleaving the resulting elongated polypeptide chain or peptidomimetic product from the porous ceramic solid support; and
(e) purifying the resulting polypeptide or peptidomimetic product.

22. The method for solid phase synthesis of polypeptides or peptidomimetics of claim 21, wherein the amino functionality is aminosilane.

23. A method for solid phase synthesis of polypeptides or peptidomimetics using a porous ceramic solid support comprising the steps of:
(a) derivatizing ceramic surface material or a three-dimensional polymer network of said porous ceramic solid support with a phenyl functionality;
(b) derivatizing the phenyl functionality to yield an appropriate functionality;
(c) coupling a compatible linker to said appropriate functionality and coupling a first amino acid or peptidomimetic moiety to said linker;
(d) coupling one or more additional amino acids or peptidomimetic moieties to said first amino acid or peptidomimetic moiety;
(e) cleaving the resulting elongated polypeptide chain or peptidomimetic product from the porous ceramic solid support; and
(f) purifying the resulting polypeptide or peptidomimetic product.

24. The method for solid phase synthesis of polypeptides or peptidomimetics of claim 23, wherein the phenyl functionality is phenyltrichloro-methylsilane.

25. A combinatorial synthesis method for preparing aromatic and heteroaromatic compound libraries using a porous ceramic solid support comprising the steps of:
(a) functionalizing ceramic surface material of said porous ceramic solid support with one or more aromatic functionalities and compatable linkers;
(b) subjecting the aromatic functionalities to reactions which result in synthesis of said aromatic and heteroaromatic compounds; and
(c) cleaving and isolating the resulting compounds.

26. A combinatorial synthesis method for preparing aromatic and heteroaromatic compound libraries using a porous ceramic solid support, wherein said porous ceramic solid support comprises pore-filled ceramic particles, comprising the steps of:
(a) functionalizing a three-dimensional polymer network within interior channels of said pore-filled ceramic particles with one or more aromatic functionalities and compatible linkers;
(b) subjecting the aromatic functionalities to reactions which result in synthesis of said aromatic and heteroaromatic compounds; and
(c) cleaving and isolating the resulting compounds.

27. The combinatorial synthesis method of claim 25 or 26, wherein the reactions of step (b) comprise one or more reactions selected from the group consisting of Friedel-Crafts alkylation with alkyl halides, alcohols, or alkenes in the presence of Lewis acids; Friedel-Crafts acylation with acid chlorides, or acid anhydrides; formulation of the arylaldehyde via the Vilsmeier reaction using activated aromatic rings, dimethylformamide and phosphorus oxychloride; Michael addition; and transition metal mediated reactions.

28. The combinatorial synthesis method of claim 27, wherein the transition metal mediated reaction is selected from the group consisting of Heck reaction, Stille coupling, and Suzuki reaction.

29. A method for generating combinatorial libraries of linkers using a porous ceramic solid support comprising the steps of:
(a) functionalizing ceramic particles of the porous ceramic solid support with chemical functionalities which permit the attachment of an organic molecule to said ceramic particles;
(b) attaching the organic molecule to said ceramic particles;
(c) subjecting the resulting attached organic molecule to reactions which result in synthesis of desired linkers;
(d) cleaving the desired linkers from the ceramic particles; and
(e) isolating the resulting linkers.

30. The method of generating combinatorial libraries of linkers of claim 29, wherein said ceramic particles are pore-filled ceramic particles.

31. A porous ceramic support for use in solid phase synthesis which comprises inert, thermally and mechanically stable ceramic particles, wherein said particles are derivatized with one or more aromatic or heteroaromatic functionalities.

32. The porous ceramic support of claim 31, wherein the ceramic particles comprise:
(a) a ceramic surface material having interior and exterior surfaces; and
(b) pores which permeate the interior of the ceramic particles.

33. The porous ceramic support of claim 32, wherein said pores are substantially filled with a three-dimensional polymer network.

34. The porous ceramic support of claim 33, wherein the aromatic or heteroaromatic functionalities derivatizing said ceramic particle are attached to the three-dimensional polymer network.
